(19) United States
(12) Patent Application Publication
(10) Pub. No.: US 2022/0017941 A1
(43) Pub. Date: Jan. 20, 2022

(54) ELECTROANALYTICAL DETERMINATION OF LEUKOCYTE ESTERASE

(71) Applicant: Board of Regents, the University of Texas System, Austin, TX (US)

(72) Inventors: Waldemar Gorski, San Antonio, TX (US); Michael Bekhit, San Antonio, TX (US)

(21) Appl. No.: 17/331,723

(22) Filed: May 27, 2021

Related U.S. Application Data
(60) Provisional application No. 63/030,481, filed on May 27, 2020.

(51) Int. Cl. C12Q 1/44; G01N 33/50; G01N 33/573; G01N 33/569

(52) U.S. Cl. CPC C12Q 1/44; G01N 33/5094; G01N 2333/918; G01N 33/56972; G01N 2333/904; G01N 33/573

(57) ABSTRACT

Disclosed are compositions and methods for detecting leukocyte esterase (LE) activity in a sample. The method can include contacting the sample with an assay sample comprising methyl pyruvate and alcohol oxidase to form a test sample, measuring H₂O₂ produced from contacting the sample and the assay sample in the test sample.

### PRIORITY

This application claims priority to U.S. Provisional Application 63/030,481 filed May 27, 2020, which is incorporated by reference in its entirety.

### STATEMENT REGARDING FEDERALLY FUNDED RESEARCH

None

### BACKGROUND

Every day, thousands of patient samples are tested in clinical laboratories for infection. However, over half of them are not infected meaning that they were unnecessarily sent for testing (Alexander B. Critical Values, 2012, 5, 6-8; Messacar et. al., J. Clin. Microbiol. 2017, 55, 715-723). More reliable screening is needed before sending samples to diagnostic labs for a more complete analysis. The benefits of such a screening will include saving resources, minimizing patient suffering, and reducing the excessive use of antibiotics and related growth of lethal antibiotic-resistant bacteria, which is one of the major challenges of modern medicine.

Historically, the diagnosis of infections in biological samples has relied on microbial cultures and non-culture methods including microscopic staining, counting leukocytes under a microscope, antibody and antigen immunoassays, and nucleic acid amplification testing (Washington et. al., Lab. Med. 1981, 12, 294-296; Carroll et. al., Am. J. Clin. Pathol. 1994, 101, 100-103; McCabe et. al., Arthritis Rheumatol. 2017, 69, 103-107; Liao et al., J. Clin. Microbiol. 2006, 44, 561-570; Pan et al., Biosens. Bioelectron. 2010, 26, 649-654; Cartwright et al., Clin. Microbiol. Newsl. 1994, 16, 33-40). These are labor-intensive techniques, which require specialized instrumentation and trained personnel.

The enzyme leukocyte esterase (LE) is an important biomarker for diagnosing and monitoring infections, a common and often devastating clinical problem. However, the development of novel LE assays has been limited (Kotani et al., Clinica Chim. Acta, 2014, 433:145-49; Murthy and Karmen, Biochem. Med. Metabol. Bio., 1988, 40:260-68; Mastropaolo and Yourno, Anal. Biochem. 1981, 115:188-93; Johnson and Schaeper, Bioconjugate Chem. 1997, 8: 76-80) and the relevant literature is dominated by the studies of clinical utility of existing LE kits and strips (McNabb et al., J. Arthroplasty 2017, 32:220-22; Colving et al., Skeletal Radiol. 2015, 44:673-77; Yadav et al., Int. J. Pharm. Bio. Sci. 2015, 6B:370-75; Ducharme et al., Can. J. Emergen. Med. 2007, 9:87-92; Bimstein et al., Pediatr. Dentist. 2004, 26:310-15), which are all based on optical assays. While such assays have been fairly useful, they often provide only semi-quantitative readings and have a limited resolution (especially, in color or opaque media).

Examples of commercially-available leukocyte esterase reagent strips are CHEMSTRIP® 9 and CHEMSTRIP® LN (both sold by Bio-Dynamics, Indianapolis, Ind.); and MULTISTIX® 2 Reagent Strips and AMES LEUKOSTIX® (both available from Ames, Division of Miles Laboratory, Elkhart, Ind.). Techniques for using these commercially-available leukocyte esterase reagent strips are well known from their use for in vitro urine analysis (e.g., Scheer, Am. J. Clin. Pathol., 1987, 87:86-93).

All of these commercially-available leukocyte esterase reagent strips contain an indoxyl carbonic acid ester which is hydrolyzed to indoxyl by leukocyte esterase. The indoxyl thus formed reacts with a diazonium compound in the strip to produce a color which indicates the presence of the leukocyte esterase. The degree of darkening of the strips is a semi-quantitative indication of the amount of leukocyte esterase present in a sample. Such colorimetric determination cannot be effectively used in turbid or color samples, e.g., bloody biological fluids, and it often requires toxic chromogenic agents, multiple liquid-handling steps, and time-consuming incubation.

### SUMMARY

Embodiments of the current invention include rapid assays and immunoassays for LE, based on electrochemical monitoring of a reaction between LE and methyl pyruvate (MP). MP is a substrate for LE and compared to several other redox substrates of LE has relatively better solubility in water (Hanson et al., ChemBioChem, 2018, 19, 1488-1491; Hanson et al., Anal. Chem., 2017, 89, 7781-7787). As illustrated in a non-limiting manner in the examples, compositions and methods of the present invention can determine the enzymatic activity of LE in human bodily fluids such as urine and saliva samples, providing rapid screening of the bodily fluids for urinary tract infection, cancers, and inflammatory gingival and periodontal diseases.

Certain embodiments are directed to a method of detecting and/or quantifying LE activity in a sample. The method can include detecting and/or quantifying LE activity in the sample electrochemically. The method can include any one of, or both of steps (a) and (b). In step (a) the sample (e.g., direct or indirect biological sample) can be contacted with an assay sample containing MP and alcohol oxidase (AOx) to form a test sample. In step (b) the amount (e.g., concentration) of H₂O₂ produced from the test sample can be measured. The amount of H₂O₂ produced in the test sample is a measure of LE activity in the sample. In some aspects, the contacting in step (a) can include contacting the sample with a dipstick containing affinity agents that bind and localize leukocyte esterase to form a contacted dipstick (an example of an indirect biological sample), and contacting the contacted dipstick with the assay sample to form the test sample. In some aspects, the affinity agents can be a LE antibody. The LE antibody can be a human anti-LE antibody. In some other aspects, the contacting in step (a) can include mixing (e.g., adding the sample to the assay sample or vice versa) to form the test sample. In some aspects, the amount of H₂O₂ in the test sample can be measured by an electrochemical method. In some particular aspects, the electrochemical method can include contacting the test sample with an electrode and measuring current flowing through the electrode. In some particular aspects, the electrode prior to contact with the test sample can be calibrated. In some aspects, the calibration can include, contacting the electrode with a calibration sample, spiking the calibration sample with aliquots (e.g. known volume and concentration) of H₂O₂ and measuring an increase of current flowing through the electrode in contact with the calibration sample due to H₂O₂ spiking. In some aspects, the calibration sample can be spiked with 20 to 100 μL aliquots of 0.1 to 0.5 mM of H₂O₂ solution, per 5 mL of calibration sample. In some particular aspects, the calibration sample can be spiked with 50 μL aliquots of 0.200 mM of H₂O₂ solution, per 5 mL of calibration sample. In some aspects, the calibration sample can be the assay sample prior to contacting the assay sample with the sample. In some other aspects, the calibration sample can be the sample prior to contacting the sample with the assay sample. In some aspects, the electrode can be a glassy carbon electrode. In some aspects, the glassy carbon electrode can contain nitrogen doped carbon nanotubes. In some aspects, the glassy carbon electrode can contain nitrogen doped carbon nanotubes dispersed in a chitosan film. In some aspects, the assay sample can contain 80 μM to 2000 μM, such as 80, 90, 100, 200, 300, 400, 500, 750, 1000, 1250, 1500, 1750, 2000 μM, including all values and ranges there between of MP. In some aspects, the assay sample can contain 25 μM to 2000 μM, such as 25, 50, 80, 90, 100, 200, 300, 400, 500, 750, 1000, 1250, 1500, 1750, 2000 μM, including all values and ranges there between of AOx. In some aspects, the sample can be contacted with the assay sample and incubated for about, or at least, 30, 35, 40, 45, 50, 55 min to 1, 1.5, 2, 2.5, 3, 3.5 hour. In certain aspects the incubation time is at least 30 min. In certain aspects the incubation time is between 30 min to 3 hours.

Certain embodiments are directed to a method for treating an infection in a subject. The method can include determining LE activity in a sample obtained from the subject according to a method of the present invention and administering a treatment of the infection to the subject if the LE activity in the sample is elevated with respect to a non-infected control. In some aspects, the subject can be a human. In some aspects, the sample can be a blood, plasma, serum, tears, urine, synovial (joint) fluid or saliva sample. In some aspects, the infection can be a urinary tract infection, or periprosthetic joint infection. In some aspects, the treatment administered can be a known treatment of infection.

Certain embodiments are directed to a leukocyte detection system. The leukocyte detection system can include an assay chamber configured to (i) contain an assay sample containing MP and AOx and (ii) be electronically coupled to an electrode and an electronic detector. In some aspects, the assay chamber can be configured to be electronically coupled to a second electrode and a third electrode forming a electrochemical cell, where the assay solution is configured to electrochemically contact the electrode, second electrode, and third electrode forming an electrochemical cell, the electrode is configured to form an working electrode of the electrochemical cell, the second electrode is configured to form a counter electrode of the electrochemical cell, the third electrode is configured to form a reference electrode of the electrochemical cell and the electronic detector is configured to detect current flowing through the working electrode. The electrode can be a glassy carbon electrode or a noble metal electrode. In some aspects, the glassy carbon electrode can contain nitrogen doped carbon nanotubes. In some aspects, the glassy carbon electrode can contain nitrogen doped carbon nanotubes dispersed in a chitosan film. In some aspects, the assay sample can contain 80 μM to 2000 μM, such as 80, 90, 100, 200, 300, 400, 500, 750, 1000, 1250, 1500, 1750, 2000 μM, including all values and ranges there between of MP. In some aspects, the assay sample can contain 25 μM to 2000 μM, such as 25, 50, 80, 90, 100, 200, 300, 400, 500, 750, 1000, 1250, 1500, 1750, 2000 μM, including all values and ranges there between of AOx. In some aspects, the counter electrode can be a platinum (Pt) wire electrode. In some aspects, the reference electrode can be a silver/silver chloride electrode. In some aspects, the system can further include a dipstick containing affinity agents that bind and localize leukocyte esterase, configured to contact a sample in which LE detection is desired and contact the assay sample after contacting with the sample. In some aspects, the affinity agents can be a LE antibody. In some aspects, the LE antibody can be a human anti-LE antibody. In some aspects, the LE antibody, such as the human anti-LE antibody can be a LE antibody known in the art.

Certain embodiments are directed to a kit containing an assay solution containing MP and AOx. In some aspects, the assay sample can contain 80 μM to 2000 μM, such as 80, 90, 100, 200, 300, 400, 500, 750, 1000, 1250, 1500, 1750, 2000 μM, including all values and ranges there between of MP. In some aspects, the assay sample can contain 25 μM to 2000 μM, such as 25, 50, 80, 90, 100, 200, 300, 400, 500, 750, 1000, 1250, 1500, 1750, 2000 μM, including all values and ranges there between of AOx. In some aspects, the kit can further include a dipstick comprising affinity agents that bind and localize leukocyte esterase. In some aspects, the affinity agents can be a LE antibody. In some aspects, the LE antibody can be a human anti-LE antibody. In some aspects, the LE antibody, such as the human anti-LE antibody can be a LE antibody known in the art.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

“Measuring leukocyte esterase”, as used herein, is detecting the presence of leukocyte esterase; or quantitatively or semi-quantitatively measuring the activity, amount or concentration of leukocyte esterase in a sample. Systems, kits, device, and/or reagents that can be used for measuring leukocyte esterase are described more fully herein.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Theoretical ICECEA amperogram for LE assay (I), and ICECEA-dipstick amperogram for LE immunoassay (II) recorded at a N-CNT electrode and based on reactions 1-3. Arrows indicate the addition of hydrogen peroxide and leukocyte esterase (LE) to a stirred assay solution of methyl pyruvate (MP) and alcohol oxidase (AOx).

FIG. 2. Effect of methyl pyruvate (MP) on the activity of LE. (A) ICECEA amperograms recorded at N-CNT electrode (−0.20 V) while adding 1.4 nM LE (43 μg L⁻¹) at 200 s to a stirred pH 7.40 PBS solution containing 25 mU AOx and (a) 1 (b) 3, (c) 5, (d) 10, (e) 20, (f) 30, (g) 40 μM MP. The current steps are due to the addition of three calibrating aliquots of H₂O₂ each yielding 2.0 μM in a solution. (B) Kinetic plot based on data in panel A. Solid line is the best non-linear regression fit of the Michaelis-Menten kinetic model to the experimental points.

FIG. 3. Assays of LE in PBS samples. ICECEA amperograms recorded at a N-CNT electrode (−0.20 V) while spiking a stirred assay solution with an increasingly concentrated enzyme aliquot to yield (a) 0 (b) 22, (c) 43, (d) 87, (e) 173, (f) 260, (g) 347, (h) 433 μg L⁻¹ LE at 200 s. The current steps are due to the addition of three H₂O₂ aliquots each yielding 7.0 μM in a solution. Assay solution (5.0 mL), pH 7.40 PBS+88 μM MP+25 mU AOx.

FIG. 4. LE activity vs. LE content and white blood cell (WBC) count in PBS samples based on data in FIG. 3. The four-color zone represents the response of a commercial LE test strip to increasing concentration of LE in a sample (trace, +, ++, +++). Assay solution (5.0 mL), pH 7.40 PBS+88 μM MP+25 mU AOx.

FIG. 5. Immunoassay of LE in a PBS solution. ICECEA-dipstick amperograms were recorded at a N-CNT electrode (−0.20 V) while dipping a d/Ab-LE antibody dipstick into an assay solution at 200 s. Each dipstick was prepared by a 30-min incubation in a PBS sample that contained (a) 0, (b) 43, (c) 75, (d) 115 (e) 150, and (f) 200 μg L⁻¹ LE. The current steps are due to the addition of three aliquots of H₂O₂ each yielding 7.0 μM in a solution. Inset: an immunosorption isotherm. The four-color zone represents the response of commercial LE test strip to increasing concentration of LE (from trace to +, ++, +++). Assay solution (3.0 mL), pH 7.40 PBS+88 μM MP+25 mU AOx.

FIG. 6. Immunoassay of LE in human (A) urine, and (B) saliva samples. ICECEA-dipstick amperograms were recorded at a N-CNT electrode (−0.20 V) while dipping a d/Ab-LE antibody dipstick into an assay solution at 200 s. Each dipstick was prepared by a 30-min incubation in a urine sample containing (a) 0, (b) 43, (c) 60, and (d) 75 μg L⁻¹ LE, and saliva sample containing (a) 0, (b) 43, (c) 75, and (d) 115 μg L⁻¹ LE. The current steps are due to the addition of three H₂O₂ aliquots each yielding 7.0 μM in a solution. Insets: immunosorption isotherms. Assay solution (3.0 mL), pH 7.40 PBS+88 μM MP+25 mU AOx. Potential of N-CNT electrode, −0.20 V.

FIG. 7. shows the ICECEA amperograms that were recorded at a N-CNT electrode at −0.20 V while the same LE aliquot was added at 200 s to the stirred pH 7.40 PBS solutions of 25 mU AO each containing 88 μM of different ester.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

A. Leukocyte Esterase Assay

The enzyme leukocyte esterase (LE) can hydrolyze methyl pyruvate (MP) to produce methanol and pyruvate (reaction 1). The methanol can be oxidized by alcohol oxidase (AOx) to produce H₂O₂ (reaction 2). Amount of H₂O₂ produced by reaction 1 and 2 is proportional to the LE activity. The amount of H₂O₂ produced can be detected electrochemically (reaction 3).

$$\begin{matrix}MP {\to }^{ LE } methanol + pyruvate & ( 1 ) \\ methonal + {O}_{2} {\to }^{ AOx } {H}_{2} {O}_{2} + formaldehyde & ( 2 ) \\ {H}_{2} {O}_{2} + 2 {H}^{+} + 2 {e}^{-} \to 2 {H}_{2} O & ( 3 )\end{matrix}$$

LE activity in a sample can be measured using an electrochemical method such as continuous enzyme assay (ICECEA) method. The sample can be contacted with an assay sample containing MP and AOx to form a test sample, H₂O₂ produced in the test sample through reaction (1) and (2) can be measured electrochemically via reaction (3) to obtain the LE activity in the sample.

H₂O₂ produced in the test sample can be measured electrochemically by contacting the test sample with an electrode and measuring a current flowing through the electrode. In some aspects, the electrode can be calibrated (e.g. internally calibrated) prior to forming a test sample. In some aspects, the electrode can be internally calibrated by contacting the electrode with a calibration sample, spiking the calibration sample with aliquots, (e.g. known amounts of H₂O₂) and measuring an increase of current flowing through the electrode in contact with the calibration sample due to H₂O₂ spiking. In some aspects, the calibration sample can be the assay sample prior to contacting the assay sample with the sample. In some other aspects, the calibration sample can be the sample prior to contacting the sample with the assay sample. In some other aspects, the calibration sample can be a third sample separate from the sample and the assay sample, and the test sample can be formed by contacting the third sample, sample and the assay sample. H₂O₂ produced in the test sample from reaction (1) and (2) can result in an increase current in the test sample over the calibration sample. In certain aspects, change of concentration of H₂O₂ in the test sample due to a volume change between the test sample and calibration sample and/or other variable factors can be factored in while calculating the LE activity in the sample. In some aspects, the electrode can be a glassy carbon electrode. In some aspects, the glassy carbon electrode can contain nitrogen doped carbon nanotubes dispersed in an anionic or cationic polymer film (e.g. chitosan). In some aspects, the assay sample and sample can be contacted by, contacting a dipstick containing affinity agents that bind and localize leukocyte esterase with the sample to form a contacted dipstick and contacting the contacted dipstick with the assay sample. In some aspects, the affinity agents can be a LE antibody. In some aspects, the affinity agents can be a human anti-LE antibody known in the art. In some other aspects, the assay sample and sample can be contacted by, adding the sample with the assay sample.

In some aspects, the MP concentration in the assay sample can be between 80 μM to 2000 μM, such as 80, 90, 100, 200, 300, 400, 500, 750, 1000, 1250, 1500, 1750, 2000 μM, including all values and ranges there between. In some aspects, the AOx concentration in the assay sample can be between 1.0, 10, 50, 100, 500, 750 and 1000, 1250, 1500, 1750, 2000 mg/L, including all values and ranges there between. In some aspects, the sample can be contacted with the assay sample and incubated for about, or at least, 30, 35, 40, 45, 50, 55 min to 1, 1.5, 2, 2.5, 3, 3.5 hour. In certain aspects the incubation time is at least 30 min. In certain aspects the incubation time is between 30 min to 3 hours.

B. Treatment Methods

Certain embodiments are directed to a method for treating an infection (e.g. a microbial infection) or an inflammation in a subject. The method can include determining LE activity in a sample obtained from the subject according to a method of the present invention and administering a treatment of the infection to the subject if the LE activity in the sample is elevated with respect to a non-infected control or reference. The non-infected control can refer to a LE activity in a non-infected state or a reference. In some aspect, the subject can be human or animal. In some aspects, the sample can be a biological fluid such as blood, plasma, serum, tears, urine, synovial (joint) fluid or saliva sample. In some aspects, the microbial infection can be a bacterial infection. In some aspects, the administered treatment of the infection can be a known treatment of the infection. In some aspects, administering a treatment of the infection can include administering an effective amount of an antibiotic to the subject. The inflammations marked by the elevated level of white blood cells (increased LE activity): (i) in synovial fluid as a marker of periprosthetic joint infection, (ii) in urine as a marker of urinary tract infection, inflammation of kidneys, prostate cancer, bladder cancer, kidney cancer, and/or (iii) in saliva as a marker of gingival and periodontal diseases. In some aspects, the infection can be a urinary tract infection or periprosthetic joint infection. In certain aspects the assays can be used to detect inflammation of kidneys, prostate cancer, bladder cancer, or kidney cancer. In other aspects, detection of LE in saliva is indicative of gingival or periodontal disease.

The assay solution, dipstick, and/or the glassy carbon electrode described herein can be incorporated into diagnostic products or kits. The diagnostic products can be used for detecting LE activity by detecting H₂O₂. In certain aspects, the diagnostic products can include at least one compound or agent useful in detecting the presence and/or concentration of H₂O₂. The term “compound or agent useful in detecting the presence of H₂O₂”, as used herein, refers to a reagent, compound, composition, or combination thereof that is changed by presence and/or concentration of H₂O₂.

Diagnostic kits can be useful for detecting LE activity by detecting H₂O₂. In certain aspects kits can include a device or apparatus or product for collecting a sample such as a biological fluid from a human or an animal being tested or diagnosed, and assay or assay device for measuring the amount of H₂O₂ released in the sample after the sample is contacted with an assay sample containing MP and AOx described herein.

The phrase “device or apparatus for collecting a sample”, as used herein, means any device or apparatus or product which is useful for removing a sample of fluid, tissue, or cells from a human or animal being tested or diagnosed without adversely affecting the ability to detect the presence of leukocyte esterase activity in the sample. Non-limiting examples of such devices include swabs, pipettes, syringes, absorbent tapes, absorbent gauzes, absorbent strips, scoops, suction bulbs, and aspirators. A kit can include one or more diagnostic products described herein.

In certain aspects the kits can be manufactured such that the sample collecting device and the assay device are separate components in the kits. The kit can include optional components to be used with the kits (e.g., test tubes for diluting samples in; bottles containing dilution fluid for diluting samples; instruction sheets; etc.) that can be combined into one package. An example of such a package is a box which is shrink wrapped with plastic.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Methyl Pyruvate as Substrates for Enzyme Leukocyte Esterase (LE)

A kinetic assay for determining the rate of a reaction between LE and MP, a MP-based coupled-enzyme assay, and an immunoassay with rapid signal transduction of interaction of MP with a LE-antibody immune complex was developed.

Methods

Reagents and Solutions. The methyl pyruvate (MP, 98%) was purchased from Alfa Aesar (Thermo Fisher). Human leukocyte suspension in 154 mM NaCl solution (cat. No. MBS173116, 86.7 μg mL⁻¹ leukocyte esterase (LE) protein, 4×10⁸ WBC mL⁻¹) and human LE ELISA kit well plate precoated with anti-human LE antibody (Ab) were purchased from MyBioSource (San Diego, Calif.). Alcohol oxidase (AOx, from Pichea Pastoris, E.C. 1.1.3.13, cat. No. 190155, 23.16 U mg⁻¹, ˜630 kDa) was from MP Biomedicals (Solon, Ohio). Chitosan (MW˜1×10⁶ Da, ˜80% deacetylation), NaOH, HCl, NaH₂PO₄.H₂O, Na₂HPO₄, and H₂O₂ (30 wt. %)) were from Sigma-Aldrich. Multi-walled carbon nanotubes doped with 1-2 at. % of nitrogen (N-CNT, 20-40 nm diameter, 50 μm average length, 8% at. Fe) were purchased from NanoTechLabs (Yadkinville, N.C.) and used as received.

The mid-stream portion of human urine sample (˜15.0 mL) was collected early in the morning in a 50.0-mL sterile centrifuge tube (Fisher Scientific, Pittsburgh, Pa.), centrifuged at 600×g for 10 min, and the supernatant was stored in a freezer at −20°. The nonstimulated whole human saliva (˜2.0 mL) was collected early in the morning before consuming any food or drink. It was centrifuged for 30 min at 10,000 g and the supernatant was stored in a 1.70-mL microcentrifuge tube (Corning, N.Y.) at −20° C.

The original suspension of human leukocytes was diluted 10 times with a pH 7.40 0.050 M phosphate buffer saline (PBS, 154 mM NaCl) solution that contained 10 vol. % DMSO and left for 10 min to lyse the leukocytes chemically. The suspension was then sonicated for 30 s (Q125 Qsonica, 20% power) to complete the lysing process. The sonication was done in 5 s intervals with 5 s rest cycles to minimize the enzyme deactivation due to solution overheating. The lysate was centrifuged at 10000×g for 5 min, and the supernatant was transferred to a 1.70-mL microcentrifuge tube (Corning, N.Y.) and stored in a freezer at −20° C. when not in use. Such a protocol yielded LE samples that had a constant esterolytic activity for at least one week.

Reagent LE strips (Siemens Multistix 5) were purchased from Siemens Healthcare Diagnostics, Inc. (Tarrytown, N.Y.). The correlation between their four-color intensity zone (trace, +, ++, +++) and LE concentration was established by using PBS solutions spiked with known amounts of LE.

The N-CNT/chitosan suspension was prepared by a 15 min sonication of 0.10 wt. % N-CNT in a 0.10 wt. % chitosan solution. The latter was prepared by dissolving chitosan flakes in a hot (80-90° C.) solution of 0.10 M HCl. The solution was cooled to room temperature, adjusted to pH˜3.5, filtered with a 0.45 μm Millex-HA syringe filter unit (Millipore Sigma), and stored at 4° C. when not in use. The concentration of H₂O₂ solutions was determined by using the absorbance at 240 nm (ε, 39.4 M⁻¹ cm⁻¹). All suspensions and solutions were prepared using 18-M)-cm deionized water that was purified with a Synergy Millipore cartridge system (Merck Millipore).

Electrochemical Measurements. The electrochemical data were collected by using a CHI 832B workstation (CH instruments) and a 3.0-mm diameter glassy carbon electrode modified with N-CNT as a working electrode. Before modification, the glassy carbon electrode was wet polished with 0.3 and 0.05 turn diameter alumina particles on an Alpha A polishing cloth (Mark V Lab, East Granby, Conn.) and cleaned by a 30 s sonications in water and methanol. The Pt wire served as a counter electrode and a reference electrode was an Ag/AgCl/3M NaCl (BASi, West Lafayette. Ind.).

The working electrode was prepared by casting a 5.0-μL aliquot of N-CNT/chitosan suspension on a glassy carbon disc and evaporating water for 2 h to form a N-CNT surface film. The polysaccharide chitosan served as a dispersant of carbon nanotubes and the adhesive holding them on the surface of electrode. The good adhesion of chitosan chains to glassy carbon assured a long-term stability (months) of such electrodes.

Before its first use, the N-CNT electrode was soaked in S solution for 2 h to hydrate the chitosan matrix and remove any loosely bound material. Afterward, the electrode was rinsed with water before use and stored capped at 4° C.

All experiments were performed at room temperature (21±1° C.). The pH 7.40 0.050 M phosphate buffer saline (PBS, 154 mM NaCl) solution was used as a background electrolyte. The experiments were repeated at least three times and the means of measurements are reported with the relative standard deviation (RSD).

Assays. The assays of LE were conducted at a N-CNT electrode by following the protocol of internally calibrated electrochemical continuous enzyme assay (ICECEA) (Zhang et. al., M.; Anal. Chem. 2013, 85, 6026-6032; Bekhit et. al., Anal. Chem. 2019, 91, 3163-3169).

The assay solution (5.00 mL) was made of pH 7.40 PBS containing both methyl pyruvate (MP) and alcohol oxidase (AOx). The current flowing through a N-CNT electrode at −0.20 V was measured continuously while a stirred assay solution was sequentially spiked with three 50-μL calibrating aliquots of H₂O₂ and one 50-μL aliquot of LE. This produced an ICECEA amperogram with the three current steps providing a calibration slope CS and an ascending linear current segment having an assay slope AS. The ICECEA amperograms shown were shortened to display only the linear portion of ascending current segment.

The AS and CS slopes were used to calculate the unit U of the enzymatic activity of LE:

$${UL}^{- 1} ( μ M {min}^{- 1} ) = \frac{AS ( μ A {s}^{- 1} ) \times 60 ( s {min}^{- 1} )}{CS ( μ A μ {M}^{- 1} )} \qquad ( 4 )$$

By definition, one unit (U) of activity was equal to the amount of LE that consumed 1.0 micromole of MP per 1.0 min in a pH 7.40 PBS solution at room temperature (21° C.).

Immunoassays. The immunoassays of LE were conducted by using the ICECEA and disposable dipsticks d/Ab (˜1.8 cm² total surface area) that were cut out of a well plate precoated with anti-human LE antibody (MyBioSource, San Diego, Calif.). The well of a dipstick was filled with 100 μL of LE sample, covered with a sealer, incubated for 30 min, and the resulting di Ab-LE dipstick was washed three times with 100-μL portions of PBS to remove weakly bound species. The time of immunocapture of LE on an antibody dipstick d/Ab (30 min) was adopted from optimized optical ELISA protocols developed for human LE. Dipping a dipstick d/Ab-LE into an assay solution (3.0 mL) during ICECEA yielded an increase in current ΔI_{LE} that was proportional to the amount of immune complex Ab-LE on a dipstick. The unknown concentration of LE was determined by using the linear immunosorption isotherm ΔI_{LE}/I_{H}_{₂}_{O}_{₂} vs. C_{LE} (μg L⁻¹), where I_{H}_{₂}_{O}_{₂} was the average of three calibrating current steps.

Recovery Experiments. The spike-and-recovery experiments were conducted by incubating dipsticks d/Ab for 30 min in three samples including the original sample (urine or saliva), sample spiked with a known LE concentration, and PBS spiked with a known LE concentration (43 and 75 μg L⁻¹ LE). Such dipsticks were then analyzed with ICECEA to obtain a ratio ΔI_{LE}/I_{H}_{₂}_{O}_{₂} for each sample. These current ratios were then used to calculate % recovery of LE.

$$\% recovery = \frac{\begin{matrix}{( Δ {I}_{LE} / {I}_{{H}_{2} {O}_{2}} )}_{( sample spiked w / LE )} - \\ {( Δ {I}_{LE} / {I}_{{H}_{2} {O}_{2}} )}_{( original sample )}\end{matrix}}{{( Δ {I}_{LE} / {I}_{{H}_{2} {O}_{2}} )}_{( PBS spiked w / LE )}} \times 100 \qquad ( 5 )$$

Results and Discussion

Design of Assays and immunoassays for LE. The design was based on coupling the enzymatic reaction of leukocyte esterase (LE), which cleaves the ester bond of methyl pyruvate (MP) releasing methanol:

$$MP {\to }^{ LE } methanol + pyruvate \qquad reaction 1$$

to a second enzymatic reaction:

$$methonal + {O}_{2} {\to }^{ AOx } {H}_{2} {O}_{2} + formaldehyde \qquad reaction 2$$

that uses alcohol oxidase (AOx) to oxidize the released methanol and produce H₂O₂. The latter can be detected via the electroreduction at a nitrogen-doped carbon nanotube (N-CNT) electrode:

$${H}_{2} {O}_{2} + 2 {H}^{+} + 2 {e}^{-} {\to }^{@ N \text{-} CNT} 2 {H}_{2} O \qquad reaction 3$$

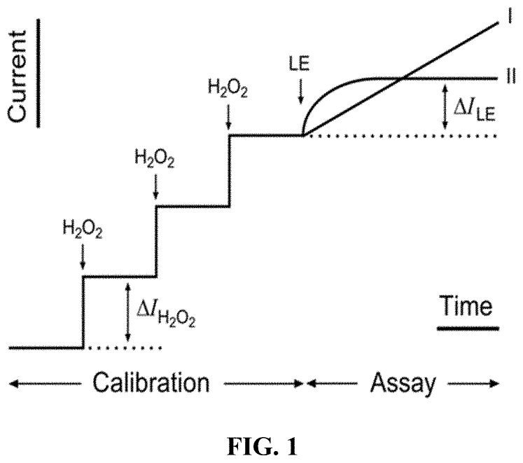

allowing to measure the kinetics of reaction of ester bond cleavage of MP, which provides the information about the enzymatic activity of LE in a sample. The N-CNT electrode has been recently shown to act as fast-responding H₂O₂ sensor with a low limit of detection (0.50 μM) at a low detection potential (−0.20 V) (Bekhit et. al., Anal. Chem. 2019, 91, 3163-3169). Such a low potential minimized interferences from other redox active species potentially present in real-life samples (e.g. vitamin C, uric acid, acetaminophen). Attempts to directly assay LE by using only a reaction 1 failed because its redox active product (methanol) yielded unstable analytical signal (anodic current) at conventional carbon and metal electrodes. FIG. 1 shows the anticipated shapes of ICECEA amperograms based on reactions 1-3. The stirred assay solution composed of MP and AOx is initially spiked with H₂O₂, which is reduced at a N-CNT electrode yielding the calibrating current steps. This is followed by the addition of LE to a solution, which triggers reaction 1 and 2 yielding extra H₂O₂ and, hence, extra current. In the assay, this extra current appears as an ascending linear segment (trace I), with a slope proportional to the initial rate of reaction 1, i.e. enzymatic activity of LE. In the immunoassay, the extra current has a form of ascending line with a plateau (trace II), which is proportional to the amount of antibody-bound LE on a dipstick that is immersed into an assay solution. The current levels off because of the saturation of the limited amount of LE on a dipstick with a substrate. These considerations are valid assuming that (i) the reaction 1 is the rate determining step, (ii) the production of H₂O₂ in reaction 2 is proportional to production of methanol in reaction 1, (iii) the antibody-bound LE retains a degree of its enzymatically active conformation, and (iv) the amount of LE immunosorbed on a dipstick is proportional to the LE content in an original sample.

Kinetic Testing. The hypothesis formulated above was tested first by determining the kinetics of reaction 1. To ensure that the reaction was rate-limiting, the excess of AOx (25 mU) over LE (3 mU) was used. In addition, the concentration of H₂O₂ was kept within a linear range of calibration plot (0.50-40 μM) to avoid the non-linearity of electrode response to H₂O₂ from affecting kinetic measurements.

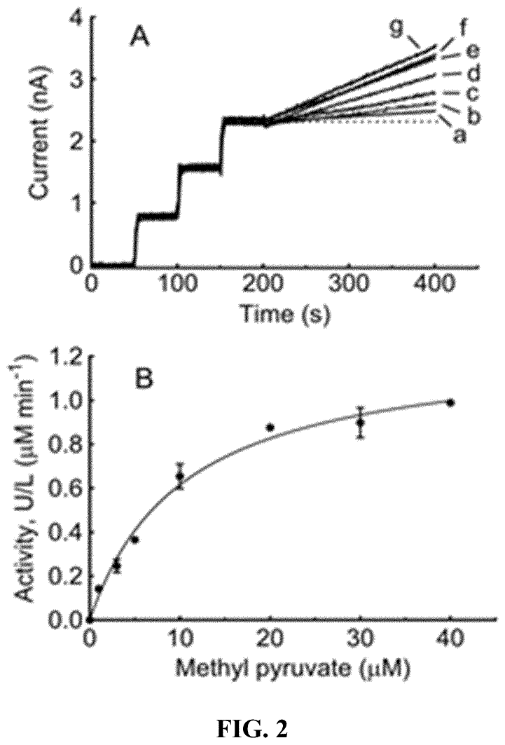

FIG. 2A shows the actual ICECEA amperograms, which have a shape anticipated for LE assay (FIG. 1, trace I) with ascending current segments recorded after the addition of 1.4 nM LE to an assay solution at 200 s. Significantly, the steady current steps at <200 s showed that the non-enzymatic hydrolysis of MP was slow on the experimental time scale and did not interfere with such LE assays. The slope of current segments at >200 s increased with the concentration of MP in a solution. These observations indicated that the MP acted as LE substrate and allowed for a femtomole detection of LE via fast coupling of reactions 1 and 2 via methanol.

FIG. 2B displays the kinetic plot that was constructed based on the ICECEA amperograms. It shows that the enzymatic activity of LE increased up to 20 μM MP and levelled off afterwards resembling the Michaelis-Menten kinetic model. Fitting this model to the experimental points yielded the turnover number k_{cat}=15 s⁻¹ and specificity constant k_{cat}Kₘ⁻¹=2.3×10⁶ M⁻¹ s⁻¹ (RSD<10%). These numbers compare well with those for other recently reported redox substrates of LE (k_{cat}=3-6 s⁻¹, k_{cat}Kₘ⁻¹=3-6×10⁵ M⁻¹ s⁻¹) (Hanson et al., ChemBioChem, 2018, 19, 1488-1491).

Assay of LE. The analytical merit of the coupling of reactions 1 and 2 via methanol was evaluated by using the MP to assay LE in PBS samples. To conduct the assay under the zero-order kinetics, the high concentration of MP was selected (88 μM, >10Kₘ).

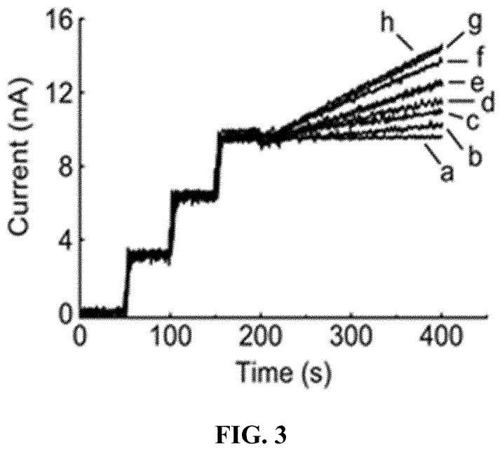

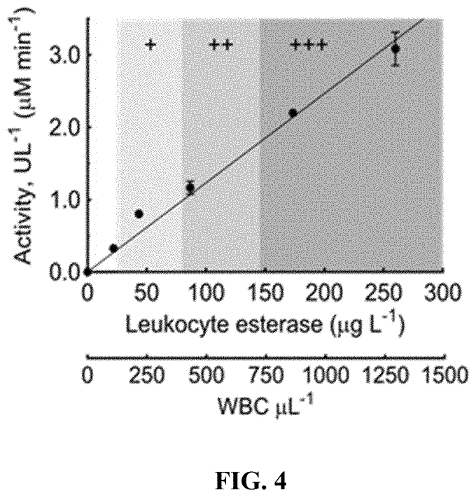

FIG. 3 shows the ICECEA amperograms that were recorded at a N-CNT electrode while different amounts of LE were added to the stirred assay solutions at 200 s. As expected, the more LE added the steeper was the ascending current segment. The slope of such segments, together with equation 4, provided the enzymatic activity of LE in each solution. FIG. 4 shows that the plot of LE activity vs. LE concentration was linear up to 260 μg L⁻¹ (R²=0.985). Its slope was equal to 12 U mg⁻¹, which represented the average specific activity of LE reacting with MP. Significantly, the plot documents that the new assay discerns differences in LE content within a one-color zone and covers all clinically relevant zones (trace, +, ++, +++) of a colorimetric LE strip.

The limit of detection (LOD) was equal to 0.71 nM LE (22 μg L⁻¹) when measured as a minimum concentration of LE that yielded the current difference I_{400 s}−I_{200 s} higher than 3 times peak-to-peak noise of the last current step. Such a LOD was similar to that for the other redox substrates of LE (5-9 μg L⁻¹), which were recently synthesized (Hanson et al., ChemBioChem, 2018, 19, 1488-1491; Hanson et al., Anal. Chem., 2017, 89, 7781-7787). The key advantage of MP over those other LE substrates is its good solubility in water and commercial availability. The signal reproducibility was investigated with the three independently prepared N-CNT electrodes. When used to measure the activity of 2.8 nM LE solution, they gave the RSD equal to 4.5% documenting a good precision of a developed assay.

Immunoassay of LE. The immunoassay was developed in order to separate LE from a sample and, thus, avoid potential interferences from the often-complex matrix of a real-life sample. To this end, the antibody dipstick d/Ab was incubated in a LE sample to sorb LE on the dipstick in the form of Ab-LE immunocomplex. The hypothesis was that the LE immobilized on such a d/Ab-LE dipstick would retain its enzymatic activity, which would then be quantified by ICECEA using MP and AOx. The rationale was that this would significantly shorten the analysis time by avoiding a laborious immobilization of detection antibody that is a part of a classical sandwich ELISA.

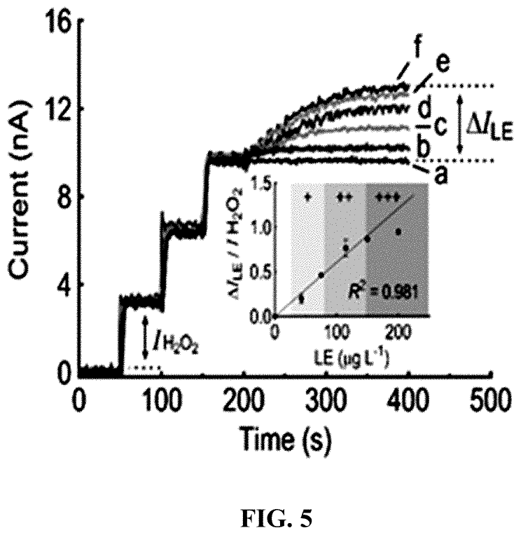

Experiments were conducted first with the PBS solutions of LE. The d/AB dipstick was incubated for 30 min in such a solution and washed before using it to collect an ICECEA amperogram. FIG. 5 shows that dipping such a d/Ab-LE dipstick into an assay solution at 200 s caused an increase in current ΔI_{LE}, which indicated the expected enzymatic activity of Ab-LE immune complex. Apparently, the LE preserved a degree of its active conformation after binding to Ab.

The higher the LE content in a PBS solution the larger was A/LE. The control experiments showed that the immersion of LE-free dipstick (d/Ab) into an assay solution did not cause any noticeable change in current (FIG. 5, trace a). Also, no change in current was observed when bare dipsticks (no Ab), which were previously incubated for 30 min in a 3.2 nM (100 μg L⁻¹) LE solution, were immersed into an assay solution. These observations indicated that the signal ΔI_{LE} was not elicited by an antibody dipstick d/Ab itself and was not affected by a non-specific adsorption of LE. Apparently, the necessary condition for the appearance of signal was the presence of Ab-LE immune complex on the surface of a dipstick.

The signal ΔI_{LE} was normalized with respect to the average current step I_{H}_{₂}_{O}_{₂} and plotted as a function of LE content in a PBS solution (FIG. 5, inset). Such an immunosorption isotherm was linear up to 150 μg L⁻¹ (R²=0.981), which was less than the upper limit of linearity for an assay calibration plot (260 μg L⁻¹, FIG. 4). This can be ascribed to the limited amount of anti-LE antibody on a d/Ab dipstick. Notably, the LODs of both immunoassay and assay were practically the same (22 μg L⁻¹) and higher than 16 ng L⁻¹ reported for sandwich ELISA (MyBioSource.com. Human Leukocyte Esterase (LE) ELISA Kit: Instruction Manual. 2019.). However, the present immunoassay is less labor intensive and cuts the required sample incubation time from over 4 h (sandwich ELISA) to 0.5 h (ICECEA-dipstick) (MyBioSource.com. Human Leukocyte Esterase (LE) ELISA Kit: Instruction Manual. 2019.).

The ICECEA-based immunoassay proposed here is based on a human anti-LE antibody that is used in the commercial sandwich ELISA kits for LE. Therefore, it has the same degree of specificity with no significant cross-reactivity or interference from the analogues.

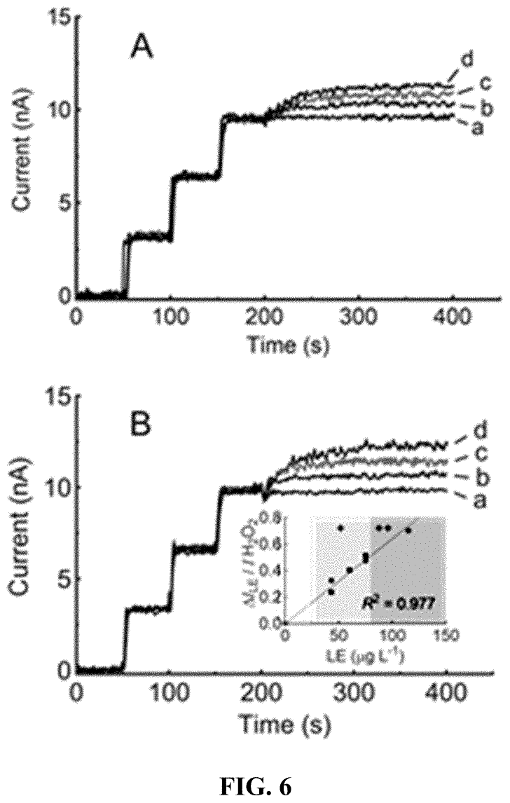

Immunoassays of LE in Human Urine and Saliva. FIG. 6 shows the ICECEA-dipstick amperograms for the determination of LE in human urine (panel A) and saliva (panel B) samples. At 200 s, the antibody dipsticks that were incubated is such samples for 30 min were immersed in a stirred assay solution. This caused the increase in current that was directly proportional to the concentration of LE in an original sample. The LOD was the same as that found for LE in PBS solution (22 μg L⁻¹). Furthermore, the immunosorption isotherm made of the data points for both urine and saliva samples (FIG. 6B, inset) was linear with a slope of 6.4×10⁻³ μg L⁻¹, which is practically the same as that for PBS samples of LE (6.2×10⁻³ μg L⁻¹, FIG. 5). This indicated that the ICECEA-dipstick immunoassay for LE was largely insensitive to the nature of sample matrix including urine, saliva, and PBS, which should simplify such measurements of LE in real-life samples.

To further validate the proposed immunoassay, the spike-and-recovery experiments were performed by recording the ICECEA-dipstick amperograms for the LE-free and LE-spiked urine and saliva samples. Such an analysis, based on equation 5, showed that the recovery was equal to 104 and 102% for urine samples spiked with 43 and 75 μg L⁻¹ LE, respectively. In the case of saliva samples spiked with 43 and 75 μg L⁻¹ LE, the recovery was 102 and 99%, respectively. This revealed a good measurement accuracy and efficient immunoseparation of LE from the complex bodily fluids (e.g. saliva contains over 2000 proteins other than LE).

It is worth noting that all of the data presented here were collected at one N-CNT electrode, which yielded reproducible ICECEA amperograms for at least 6 months. This could be ascribed to a good dispersion of carbon nanotubes in a chitosan film and its strong adhesion to the glassy carbon surface. In addition, the in-situ calibration of ICECEA reduced the errors caused by a drifting activity of electrode surface increasing the accuracy and precision of such measurements.

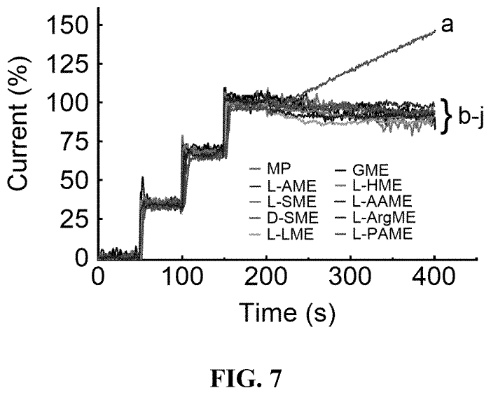

Control Experiments with Small Esters Other Than Methyl Pyruvate. FIG. 7 shows the ICECEA amperograms that were recorded at a N-CNT electrode at −0.20 V while the same LE aliquot was added at 200 s to the stirred pH 7.40 PBS solutions of 25 mU AO each containing 88 μM of different ester. The selected esters had a common structural motif made of O═C—OCH₃ group (Table 1). Among the ten esters tested, only methyl pyruvate (trace a) produced the ascending current segment starting at 200 s. This indicated that methyl pyruvate was recognized by LE as its substrate and could be used for the determination of LE. The lack of the recognition of other esters (traces b-j) could be ascribed to the combination of structural and electronic effects of their substituents and the lack of the proper orientation necessary for the binding interactions with specific amino acid residues in the catalytic site of LE. The data in FIG. 7 underline the uniqueness of the methyl pyruvate-based method for the quantification of LE as a biomarker of infection.

TABLE 1
SELECTED ESTERS USED IN SCREENING EXPERIMENTS WITH LE.

| Name of ester | Abbreviation | Structure |
|---|---|---|
| Methyl pyruvate | MP | 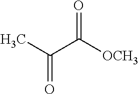 |
| L-Alanine methyl ester hydrochloride | L-AME | 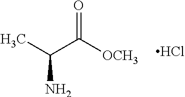 |
| L-Serine methyl ester hydrochloride | L-SME | 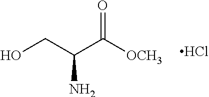 |
| D-Serine methyl ester hydrochloride | D-SME | 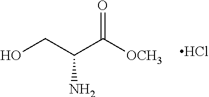 |
| L-Leucine methyl ester hydrochloride | L-LME | 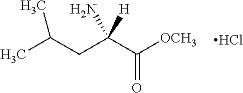 |
| Glycine methyl ester hydrochloride | GME | 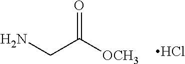 |
| L-Histidine methyl ester hydrochloride | L-HME | 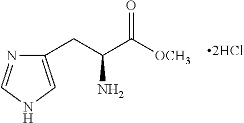 [SMILES: COC(=O)[C@@H](N)Cc1c[nH]cn1 ; "·2HCl"] |
| L-Aspartic acid dimethyl ester dihydrochloride | L-AAME | 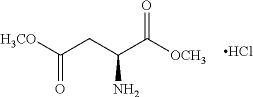 [SMILES: COC(=O)C[C@H](N)C(=O)OC ; Cl] |
| L-Arginine methyl ester dihydrochloride | L-ArgME | 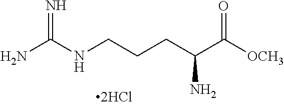 [SMILES: COC(=O)[C@@H](N)CCCNC(=N)N] |
| L-Phenylalanine methyl ester dihydrochloride | L-PAME | 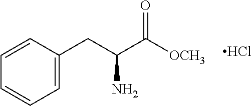 [SMILES: COC(=O)[C@@H](N)Cc1ccccc1 ; Cl] |

1. A method for detecting leukocyte esterase (LE) activity in a sample, the method comprising:
(a) contacting the sample with an assay sample comprising methyl pyruvate and alcohol oxidase to form a test sample; and
(b) measuring concentration of H₂O₂ produced from contacting the sample and the assay sample in the test sample.

2. The method of claim 1, wherein contacting in step (a) comprises, contacting the sample with a dipstick comprising affinity agents that bind and localize leukocyte esterase to form a contacted dipstick, and contacting the contacted dipstick with the assay sample to form the test sample.

3. The method of claim 1, wherein contacting in step (a) comprises adding the sample with the assay sample to form the test sample.

4. The method of claim 1, wherein the H₂O₂ concentration produced in the test sample is measured by an electrochemical method.

5. The method of claim 4, wherein the electrochemical method comprises contacting the test sample with an electrode and measuring current flowing through the electrode.

6. The method of claim 5, wherein the electrode is calibrated prior to contacting the test sample with the electrode, comprising contacting the electrode with a calibration sample, spiking the calibration sample with aliquots of H₂O₂ and measuring an increase in current flowing through the electrode in contact with the calibration sample.

7. The method of claim 6, wherein the calibration sample can be spiked with 50-μL aliquots of 0.200 mM H₂O₂ solution, per 5 ml of calibration solution.

8. The method of claim 6, wherein the calibration sample is the assay sample prior to contacting the assay sample with the sample, or the sample prior to contacting the sample with the assay sample.

9. The method of claim 5, wherein the electrode is a glassy carbon electrode.

10. The method of claim 9, wherein the glassy carbon electrode comprises nitrogen doped carbon nanotubes.

11. The method of claim 9, wherein the glassy carbon electrode comprises nitrogen doped carbon nanotubes dispersed in a chitosan film.

12. The method of claim 1, wherein the assay solution comprises 10 mg/L to 2000 mg/L of methyl pyruvate.

13. The method of claim 1, wherein the assay solution comprises 10 mg/L to 2000 mg/L of alcohol oxidase.

14. A method for treating an infection in a subject, the method comprising:
determining LE activity in a sample obtained from the subject, according to claim 1;
administering a treatment of the infection to the subject if the LE activity in the sample is elevated with respect to a non-infected control.

15. The method of claim 14, wherein the subject is a human.

16. The method of claim 14, wherein the sample is a blood, plasma, serum, tears, urine, synovial (joint) fluid or saliva sample.

17. The method of claim 14, wherein the infection is an urinary tract infection, or periprosthetic joint infection.

18. An leukocyte detection system comprising:
an assay chamber configured to (i) contain an assay solution comprising methyl pyruvate and alcohol oxidase and (ii) be electronically coupled to an electrode and an electronic detector.

19. The system of claim 18, wherein the assay chamber is configured to be electronically coupled to a second electrode and a third electrode forming a electrochemical cell, wherein the assay solution is configured to electrochemically contact the electrode, second electrode and third electrode forming an electrolyte of the electrochemical cell, the electrode is configured to form an working electrode of the electrochemical cell, the second electrode is configured to form a counter electrode of the electrochemical cell, the third electrode is configured to form a reference electrode of the electrochemical cell and the electronic detector is configured to detect current flowing through the working electrode.

20.-26. (canceled)

27. A kit comprising an assay solution comprising methyl pyruvate and alcohol oxidase and a dipstick comprising affinity agents that bind and localize leukocyte esterase.

28.-30. (canceled)
